(19) United States
(12) Patent Application Publication
(10) Pub. No.: US 2014/0303251 A1
(43) Pub. Date: Oct. 9, 2014

(54) Methods Of Treating Acute Kidney Injury With Retinoic Acid

(71) Applicants: Terry B. Strom, Brookline, MA (US); Savithri Balasubramanian, Brookline, MA (US); Benjamin D. Humphreys, Brookline, MA (US)

(72) Inventors: Terry B. Strom, Brookline, MA (US); Savithri Balasubramanian, Brookline, MA (US); Benjamin D. Humphreys, Brookline, MA (US)

(73) Assignees: Beth Israel Deaconess Medical Center, Inc., Boston, MA (US); The Brigham and Women's Hospital, Inc., Boston, MA (US)

(21) Appl. No.: 13/981,802

(22) PCT Filed: Jan. 31, 2012
(86) PCT No.: PCT/US2012/023233

§ 371 Date: Jun. 5, 2014

Related U.S. Application Data
(60) Provisional application No. 61/437,971, filed on Jan. 31, 2011.

(51) Int. Cl. A61K 31/203; A61K 45/06

(52) U.S. Cl. CPC A61K 31/203; A61K 45/06
USPC 514/559; 435/375

(57) ABSTRACT

The present invention provides, in one embodiment, a method of inhibiting apoptosis in kidney epithelial cells after acute injury by modulating/inhibiting Nur77 induction/expression or function/activity in the cells. The invention further provides a method of preventing or treating an acute kidney injury in a subject in need thereof, comprising administering to the subject an effective amount of a retinoic acid or retinoic acid derivative, and a method of preserving a kidney for transplant, comprising contacting the kidney with a solution that comprises a retinoic acid or a, retinoic acid derivative.

### RELATED APPLICATION

This application claims the benefit of U.S. Provisional Application No. 61/437,971, filed on Jan. 31, 2011. The entire teachings of the above application are incorporated herein by reference.

### GOVERNMENT SUPPORT

This invention was made with government support under grants DK073628, DK088923 and P01 A73748 awarded by the National Institutes of Health. The government has certain rights in the invention.

### BACKGROUND OF THE INVENTION

Acute kidney injury (AKI) is common, costly and independently associated with increased risk of death¹. Patient outcomes are directly related to AKI severity, including even minor changes in serum creatinine². More severe AKI is an independent risk factor for death with mortality rates generally exceeding 30% and often exceeding 50% when it occurs in the setting of trauma, surgery or multiple organ dysfunction³. After acute injury, tubular epithelial cells die through both necrotic and apoptotic mechanisms, and cells that survive the insult dedifferentiate and proliferate to reconstitute a functioning nephron⁴. Strategies to increase the fraction of epithelial cells that survive the initial insult should be therapeutic, by providing a larger mass of epithelial progenitors during the repair phase.

Acute kidney injury remains a major health problem with few therapeutic options. Accordingly, there is a considerable need for new therapies and therapeutic agents that are effective for the treatment of AKI.

### SUMMARY OF THE INVENTION

As shown herein, Nur77 is rapidly and strongly induced in proximal tubule epithelia by acute kidney injury using in vivo and in vitro experimental models. In Nur77 knockout studies, Nur77 induction after acute kidney injury promotes epithelial apoptosis, and the absence of Nur77 reduces apoptosis, prevents pro-inflammatory cytokine induction and ameliorates histologic damage, leukocyte infiltration and renal function. In addition, as shown herein, 9-cis-retinoic acid reduces Nur77 induction and ameliorates acute kidney injury in wild type mice in vivo, and this protection is Nur77-dependent in vitro. Thus, the experiments provided herein support retinoid-based modulation of Nur77 induction as a novel strategy for the treatment of acute kidney injury. Such injuries can include hypoxia-induced injuries, ischemic reperfusion injuries, toxic or septic injuries.

Accordingly, the present invention provides, in one embodiment, a method of inhibiting apoptosis in kidney epithelial cells after acute injury by modulating/inhibiting Nur77 induction/expression or function/activity in the cells. In a particular embodiment, Nur77 induction/expression is inhibited by contacting the cells with retinoic acid, or a derivative thereof.

In another embodiment, the invention provides a method of preventing or treating an acute kidney injury in a subject in need thereof, comprising administering to the subject an effective amount of a retinoic acid or retinoic acid derivative. In a particular embodiment, the acute kidney injury is an ischemic renal injury.

In a further embodiment, the invention provides a method of preserving a kidney for transplant, comprising contacting the kidney with a solution that comprises a retinoic acid or a retinoic acid derivative, such as a cis- or trans-retinoic acid.

The present invention provides an important new therapeutic target and effective therapeutic agents for the treatment of acute kidney injury and related conditions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Induction and localization of Nur77 in the kidney during renal IRI. (A) NR4a family members, predominantly Nur77, are induced after renal IRI (B) Nur77 gene expression is highly induced in renal medulla, site of the most severe epithelial injury in the IRI model.

FIG. 2: (A) Nur77 is highly induced in primary proximal tubule epithelial cultures one hour after ischemia-reperfusion and returns to baseline by three hours (n=3). (E-G) Nur77 is expressed in tubular epithelial cells after IRI in wild type kidneys, as assessed by in situ hybridization. (B-D) Nur77 gene expression was not detected either in sham operated or IRI induced Nur77⁻⁻ kidneys.

FIG. 3: Nur77^{-/-} mice are protected from renal ischemia-reperfusion injury. (A) Serum blood urea nitrogen*** (BUN) and (B) creatinine*** were significantly lower in Nur77^{-/-} mice (n=9) 24 hours after IRI compared to wild type littermate controls (n=8), ***P<0.0001. (C) Kidney injury as scored by tubular dilatation, necrosis, casts and loss of brush border was severe in Nur77^{+/+} as compared to Nur77^{-/-} mice; (Nur77^{+/+} vs. Nur77^{-/-}: 4±0.2 vs. 2.4±0.44 (AVE±SEM) (n=9 for Nur77^{+/+} and n=11 for Nur77^{-/-}) P<0.005 (D) Less tubular cell apoptosis was observed in Nur77^{-/-} mice as compared to controls assessed by Tunel assay. (E) Quantitation of Tuner nuclei showed a 3 fold reduction in epithelial apoptosis in Nur77^{-/-} kidneys, **P=0.001 (n=4 for Nur77^{+/+} and n=6 for Nur77^{-/-}) (F) Reduced neutrophil infiltration in Nur77^{-/-} kidneys compared to Nur77^{+/+} was detected. (G) There is a 50% reduction in neutrophil infiltration into the cortico-medullary junction of the kidneys in Nur77^{-/-} (n=4 for Nur77^{+/+} and n=6 for Nur77^{-/-}) **P<0.004.

FIG. 4: Reduced expression of proinflammatory cytokines and chemokines in Nur77^{-/-} kidney tissues upon IRI. (A-H) Cytokine and pro-inflammatory gene expression in kidney tissues from Nur77^{+/+} or Nur77^{-/-} kidneys was measured by qPCR. (A) CXCL2, (B) CCL20, (C) CXCL10 (D) IL-1 beta, (E) TGFβ (F) IL-6 (G) CSF-1 and (H) ICAM-1 in Nur77^{+/+} and Nur77^{-/-} kidneys is presented as fold change over the sham control (Ave±SEM, n=6 for each). (I) Induction of pro-inflammatory milieu is not completely dependent on the onset of apoptosis as noted by very early induction of proinflammatory chemokines such as Cxcl2 and Ccl20 upon hypoxia reoxygenation in RPTECs, as measured by qPCR (Ave+SEM, n=3 each) and (J) production of Cxcl2 is independent of the onset of apoptosis, as evident from the lack of costaining with cleaved caspase 3.

FIG. 5: Effect of 9-cis-RA treatment in the in vitro IRI model. In primary cultures of renal proximal tubule epithelial cells (RPTECs) from either Nur77^{+/+} or Nur77^{-/-} mice, ischemia-reperfusion induced the expression of (A) Nurr77, (B) Nor-1 and (C) Nurr1, and 9-cis-RA treatment attenuated the induction of all three NR4a genes as measured by qPCR. (D, E) Expression of CXCL2 and CCL20 after IRI was reduced by 9-cis-RA treatment of Nur77^{+/+} RPTEC cultures. Expression of these same genes in Nur77^{-/-} cultures was lower compared to Nur77^{+/+} cells, and 9-cis-RA did not substantially reduce the expression level further, as measured by qPCR (Ave±SEM, n=3 each).

FIG. 6: 9-cis-RA dampens Nur77 gene expression and ameliorates renal IRI in vivo. Upon exposure to 9-cis-RA (10 mg/kg body weight) prior to the induction of IRI, (A) the gene expression level of Nur77 was significantly diminished. Nor-1 and Nurr-1 levels were also reduced in the 9-cis-RA treated mice kidneys as compared to the vehicle treated group. (B) BUN*** and (C) serum creatinine** were significantly lower in mice that received 9-cis-RA (n=11) compared to vehicle (n=9), ***P<0.0001 and **P<0.002. (D) Upon IRI, fewer apoptotic cells were detected in kidneys from mice treated with 9-cis-RA as compared to the vehicle treated group (n=6 for each group and P<0.001) (E) as were numbers of infiltrating neutrophils (n=6 for each group and P<0.001) (F) Tubular injury scoring reveals that the 9-cis-RA treated mice kidneys were less injured as compared to the vehicle control. (G) Gene expression level of CXCL2 was greatly diminished in the 9-cis-RA treated mice as compared to the vehicle treated mice (n=6 for each group, P<0.02).

FIG. 7: 9-cis-RA treatment does not alter the localization pattern of Nur77 during IRI. Nur77 expression and localization pattern was ascertained by immunochemistry during IRI. (A) Nur77 staining was observed in the nucleus immediately after reperfusion, which at 24 h post IRI was found predominantly in the cytoplasm. (B) Localization pattern of Nur77 is unaltered with or without the treatment of 9 cis-RA, 24 h post IRI.

FIG. 8: Purity of primary cultures of renal proximal tubular epithelial culture, ascertained by cytokeratin staining.

FIG. 9: Induction of Nur77 in immortalized human RPTEC line, HK2. Nur77 expression is triggered by ischemia reperfusion injury induced by a cocktail of Antimycin (10 uM), 2-Deoxy Glucose (10 mM) and A23187 (2 uM) for 1 hour and reperfused for indicated periods of time.

FIG. 10: Expression kinetics of Retinoid X Receptor (RXR) and Retinoic Acid Receptor (RAR) family members during IRI.

FIG. 11: Transcript level of HIF-1 alpha is unchanged upon treatment with 9-cis-RA, prior to the induction of IRI.

FIG. 12: Involvement of Bcl2 family proteins in Nur77 mediated renal injury. (A) Expression of Nur77 during renal IRI results in the pronounced exposure of BH3 domain of Bcl2 predominantly in the inner medulla of wild type kidneys as compared to the Nur77KO or sham operated kidneys. (B) Increased protein levels of the pro-apoptotic protein, Bcl-xS in the kidney tissues upon IRI, which is diminished in the Nur77 deficient kidneys. Arrows indicate the long and short forms of Bcl-x protein. N.S: non-specific bands.

### DETAILED DESCRIPTION OF THE INVENTION

After acute kidney injury, epithelial cells die both through necrotic and apoptotic mechanisms and there is intense interest in understanding the signaling pathways that regulate these cell death pathways in order to design new therapeutic strategies^{5,6}. While progress has been made in defining the execution phase of kidney apoptotic cell death, the upstream signaling pathways that initiate epithelial apoptosis after injury are poorly understood. Nur77 represents a strong candidate for such an apoptotic switch protein after injury. Hypoxia induces Nur77 expression via hypoxia-inducible factor-1a (HIF-1a)⁷, a transcription factor strongly induced by ischemic renal injury⁸, and Nur77 itself enhances HIF-1a transcriptional activity⁹. Originally identified as an immediate early gene transiently induced by serum or growth factors¹⁰), Nur77 is a potent proapoptotic inducer during thymocyte selection¹¹ and in epithelial cancer cells exposed to antineoplastic agents, including lung¹², prostate¹³ and colon¹⁴ through direct interaction with Bcl-2. These observations have led to development of Nur77 agonists as potential anticancer treatments.¹⁵

While Nur77 binds DNA and directly activates gene transcription; its function also depends on subcellular localization: in the nucleus Nur77 functions as an oncogenic survival factor whereas Nur77 translocation to mitochondria results in direct binding to Bcl-2 and conversion to an apoptotic phenotype¹⁶. Proapoptotic Nur77 nuclear export can be mediated by heterodimerization with retinoid X receptor (RXR), a process that is inhibited by retinoic acid ligand binding to RXR¹⁷. Retinoids also negatively regulate Nur77 by potently suppressing its transcriptional induction ¹⁸. The observation that retinoic acid can ameliorate toxin-induced AKT suggests the possibility that this effect might be mediated by inhibition Nur77¹⁹.

As described herein, it is demonstrated for the first time that Nur77 is rapidly and strongly induced in proximal tubule epithelia by acute kidney injury using in vivo and in vitro experimental models. In Nur77 knockout studies described herein, it is demonstrated that Nur77 induction after acute kidney injury promotes epithelial apoptosis, and the absence of Nur77 reduces apoptosis, prevents pro-inflammatory cytokine induction and ameliorates histologic damage, leukocyte infiltration and renal function. Importantly, it is shown that 9-cis-retinoic acid reduces Nur77 induction and ameliorates acute kidney injury in wild type mice in vivo, and this protection is Nur77-dependent in vitro. Retinoid-based modulation of Nur77 induction offers a novel strategy for the treatment of acute kidney injury.

Unless defined otherwise, all technical and scientific, terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

In one embodiment, the invention provides a method of inhibiting apoptosis in kidney epithelial cells after acute injury. Apoptosis may be inhibited by modulating (e.g., inhibiting) Nur77 induction/expression (e.g., Nur77 transcription, heterodimerization of Nur77 with retinoid X receptor) or function/activity (e.g., translocation from nucleus to cytoplasm) in the kidney cells. In a preferred embodiment, the kidney cells are proximal tubule epithelial cells.

The acute injury can be a hypoxia-induced injury. In such injuries, the hypoxia may be caused, for example, by an ischemia reperfusion injury (e.g., a renal ischemia reperfusion injury).

Nur77 induction/expression may be inhibited by contacting the cells with retinoic acid, or a derivative thereof. The retinoic acid (RA) may be all-trans-retinoic acid or a cis-retinoic acid (e.g., 7-cis-retinoic acid, 9-cis-retinoic acid, 11-cis-retinoic acid, 13-cis-retinoic acid). In a particular embodiment, Nur77 induction/expression is inhibited by contacting the cells with 9-cis-retinoic acid.

Derivatives of retinoic acid include, but are not limited to, amides and esters of retinoic acid, as well as retinamide/retinyl ester mimics. All-trans isomeric form of retinamides and retinyl esters are characterized by Structural Formula (1) shown below:

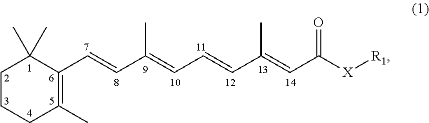

where X is —NR′— or —O—; and R₁ is a substituted or unsubstituted aliphatic or aryl group, or a substituted or unsubstituted non-aromatic heterocyclic group; and R′ is hydrogen or a substituted or unsubstituted aliphatic or aryl group, or a substituted or unsubstituted non-aromatic heterocyclic group. It is noted that other isomers of the molecules characterized by Structural Formula (1), for example, 7-cis, 9-cis, 11-cis, 13-cis, or any combination thereof, can also be used in the invention. 9-cis and 13-cis isomers are exemplified in Structural Formulas (2) and (3), where X and R₁ are as defined above:

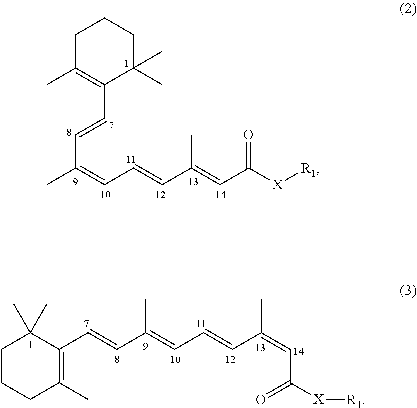

Typically, R₁ is a substituted or unsubstituted aryl or lower alkyl group. Preferably, when X is —NR′—, R₁ is a substituted aryl group, such as 4-hydroxy phenyl, 3-hydroxy phenyl or 2-hydroxy phenyl. More preferably, when X is —NR′—, R₁ is 4-hydroxy phenyl. Alternatively, when X is —O—, R₁ is preferably a substituted or unsubstituted lower alkyl group, such as methyl, ethyl, propyl or benzyl.

As used herein, the term “aliphatic group” is non-aromatic, consists solely of carbon and hydrogen and may optionally contain one or more units of unsaturation, e.g., double and/or triple bonds. An aliphatic group may be straight chained, branched, or cyclic (i.e., “cycloaliphatic”). When straight-chained or branched, an aliphatic group typically contains between about 1 and about 24 carbon atoms, more typically between about 1 and about 12 carbon atoms. Aliphatic groups are preferably lower alkyl groups or lower alkylene groups, which include C1-24 (preferably C1-C12) straight chained or branched saturated hydrocarbons. An alkyl group is a saturated hydrocarbon in a molecule that is bonded to one other group in the molecule through a single covalent bond from one of its carbon atoms. Examples of lower alkyl groups include methyl, ethyl, n-propyl, iso-propyl, n-butyl, sec-butyl and tert-butyl. An alkylene group is a saturated hydrocarbon in a molecule that is bonded to two other groups in the molecule through single covalent bonds from two of its carbon atoms. Examples of lower alkylene groups include methylene, ethylene, propylene, iso-propylene (—CH(CH2)CH2-), butylene, sec-butylene (—CH(CH3)CH2CH2-), and tert-butylene (—C(CH3)2CH2-).

As used herein, the term“aryl group” may be used interchangeably with “aryl,” “aryl ring,” “aromatic group,” and “aromatic ring. Aromatic groups include carbocyclic aromatic groups and heteroaryl rings. Examples of carbocyclic aromatic groups include phenyl, 1-naphthyl, 2-naphthyl, 1-anthracyl and 2-anthacyl. The term “heteroaryl”, “heteroaromatic”, “heteroaryl ring”, “heteroaryl group” and “heteroaromatic group” refer to heteroaromatic ring groups having five to fourteen members, including monocyclic heteroaromatic rings and polycyclic aromatic rings in which a monocyclic aromatic ring is fused to one or more other carbocyclic or heteroaromatic aromatic rings. Heteroaryl groups have one or more ring heteroatoms. Examples of heteroaryl groups include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl.

The term “non-aromatic heterocyclic group” refers to non-aromatic ring systems typically having five to fourteen members, preferably five to ten, in which one or more ring carbons, preferably one to four, are each replaced by a heteroatom such as N, O, or S. Examples of non-aromatic heterocyclic groups include 3-1H-benzimidazol-2-one, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, N-azetidinyl, 1-azetidinyl, 2-azetidinyl, N-oxazolidinyl, 2-oxazolidinyl, 4-oxazolidinyl, 5-oxazolidinyl, N-morpholinyl, 2-morpholinyl, 3-morpholinyl, N-thiomorpholinyl, 2-thiomorpholinyl, 3-thiomorpholinyl, N-pyrrolidinyl, 2-pyrrolidinyl, 3-pyrrolidinyl, N-piperazinyl, 2-piperazinyl, N-piperidinyl, 2-piperidinyl, 4-piperidinyl, N-pyrrolidinyl, 2-pyrrolidinyl, 3-pyrrolidinyl, diazolonyl, N-substituted diazolonyl, 1-pthalimidinyl, benzoxanyl, benzopyrrolidinyl, benzopiperidinyl, benzoxolanyl, benzothiolanyl, benzothianyl, tetrahydroquinolinyl, tetrahydroisoquinolinyl, azabicyclopentyl, azabicyclohexyl, azabicycloheptyl, azabicyclooctyl, azabicyclononyl, azabicyclodecyl, diazabicyclohexyl, diazabicycloheptyl, diazabicyclooctyl, diazabicyclononyl, and diazabicyclodecyl. Also included within the scope of the term “non-aromatic heterocyclic group”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring.

Suitable substituents on an aliphatic group, such as an alkyl or alkenyl group, and aryl group are those which do not substantially interfere, with the inhibiting activity of the disclosed compounds. Examples of suitable substituents include —OH, halogens (—Br, —Cl, —I, —F), —OR^{a}, —O—COR^{a}, —COR^{a}, —CN, —NCS, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{b}COR^{a}, —NHCONH₂, —NHCONR^{a}H, —NHCON(R^{a}R^{b}), —NR^{b}CONH₂, —NR^{b}CONR^{a}H, —NR′CON(R^{a}R^{b}), —C(═NH)—NH₂, —C(═NH)—NHR^{a}, C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —SH, —SR^{a}, —S(O)R^{a}, and —S(O)₂R^{a}. In addition, an aliphatic group, such as an alkyl, or alkenyl group, can be substituted with substituted or unsubstituted aryl group to form, for example, an aralkyl group such as benzyl. Similarly, an aryl group can be substituted with a substituted or unsubstituted alkyl or alkenyl group. R^{a}—R^{d} are each independently an alkyl group, aromatic group, non-aromatic heterocyclic group or —N(R^{a}R^{b}), taken together, form a substituted or unsubstituted non-aromatic heterocyclic group.

In another embodiment, the invention provides a method of preventing or treating an acute kidney injury in a subject in need thereof, comprising administering to the subject an effective amount of a retinoic acid or retinoic acid derivative. The term “acute kidney injury” or “AKI” as used herein refers to a condition that is characterized clinically by a rapid reduction in kidney function resulting in a failure to maintain fluid, electrolyte and/or acid-base homoeostasis. Acute kidney injury is commonly defined by an abrupt (e.g., within 48 h) increase in serum creatinine, resulting from an injury or insult that causes a functional or structural change in the kidney. The AKI may be caused by, for example, ischemia (e.g., ischemia reperfusion injury), sepsis and/or toxins (e.g., nephrotoxic insults such as toxic chemicals). In a particular embodiment, the AKI is an ischemic AKI. AKI includes acute renal failure (ARF), prerenal AKI (e.g., prerenal azotemia), intrinsic AKI (e.g., intrinsic renal azotemia) and postrenal AKI (e.g., postrenal azotemia). Thus, the acute kidney injury may be an ischemic injury, a toxicant-induced injury, a septic injury or a hypoxia-induced injury, or a combination thereof.

As used herein, the terms “treat,” “treating,” or “treatment,” mean to counteract an acute kidney injury (AKI) to the extent that the AKI is improved according to a clinically-acceptable standard. An improvement in an acute kidney injury can be determined according to one or more of the following clinical standards: 1) reduction in serum creatinine, 2) increase in kidney function, 3) increase in urine output, 4) decrease in blood urea nitrogen. Other markers such as the expression levels of Nur77 can be used. Clinical improvement in AKI can also be determined using one or more alternate biomarkers, such as, for example, urinary neutrophil gelatinase-associated lipocalin (NGAL), urinary interleukin 18, urinary kidney injury molecule 1 (KIM-1) and cystatin C.

“Prevent,” “preventing,” or “prevention,” as used herein, mean reducing the probability/likelihood or risk of an acute kidney injury in a subject, delaying the onset of a condition related to an acute kidney injury in the subject, lessening the severity of one or more symptoms of an acute kidney injury in the subject, or any combination thereof. In general, the subject of a preventative regimen most likely will be categorized as being “at-risk”, e.g., the risk for the subject developing an AKI is higher than the risk for an individual represented by the relevant baseline population.

The term “subject” refers to a mammal, including primates (e.g., humans), cows, sheep, goats, horses, dogs, cats, rabbits, guinea pigs, rats, mice or other bovine, ovine, equine, canine, feline, rodent or murine species. Examples of preferred subjects include, but are not limited to, humans, including humans who have, or are at risk for developing, an acute kidney injury. Examples of high-risk groups for the development of AKI include individuals having increased serum creatinine levels and reduced urine output. Further examples of high-risk groups include patients undergoing vascular surgeries and those with history of hypertension.

As used herein, an “effective amount” is an amount sufficient to inhibit (e.g., reduce, prevent) Nur77 induction/expression and/or function/activity in kidney cells of a subject. In a particular embodiment, the kidney cells are kidney epithelial cells, preferably proximal tubule epithelial cells.

A “therapeutically effective amount” is an amount sufficient to achieve the desired therapeutic or prophylactic effect under the conditions of administration, such as an amount sufficient to inhibit (e.g., reduce, prevent) an acute kidney injury. The effectiveness of a therapy can be determined by one of skill in the art using standard measures and routine methods.

The amount of the retinoic acid or derivative thereof to be administered to a subject (e.g., an effective amount, a therapeutically effective amount) can be determined by a clinician using the guidance provided herein and other methods known in the art and is dependent on several factors including, for example, the particular agent chosen, the subject's age, sensitivity, tolerance to drugs and overall well-being. For example, suitable dosages for retinoic acid or derivatives thereof can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, patient and condition is well within the abilities of one skilled in the art. Preferably, the dosage does not cause, or produces minimal, adverse side effects (e.g., immunogenic response, nausea, dizziness, gastric upset, hyperviscosity syndromes, congestive heart failure, stroke, pulmonary edema).

A therapeutically effective amount of a retinoic acid or derivative thereof can be administered alone, or in combination with one or more other therapies (e.g., vasodilation, volume replacement, blood purification and renal replacement therapy) and/or therapeutic agents useful for preventing or treating AKI, including, but not limited to, diuretics, antiobiotics, calcium and glucose/insulin. Emerging pharmacological agents for the treatment of AKI which may be used in combination with retinoic acid or retinoic acid derivatives are discussed in Jo et al., Clinical Journal of the American Society of Nephrology 2:356-365 (March, 2007), the contents of which are incorporated herein by reference, and include nonselective and selective caspase inhibitors, minocycline, guanosine, pifithrin-α, PARP inhibitors, sphingosine 1 phosphate analog, adenosine 2A agonist, α-MSH, IL-10, fibrate, PPAR-γ agonist, minocycline, activated protein C, iNOS inhibitor, insulin, activated protein C, ethyl pyruvate, recombinant erythropoietin, hepatocyte growth factor, carbon monoxide release compound and bilirubin, endothelin antagonist, fenoldopam and ANP.

The retinoic acid or derivative thereof can be administered before, after or concurrently with one or more additional therapies or therapeutic agents. In some embodiments, the retinoic acid or derivative thereof and additional therapy or therapeutic agent are co-administered simultaneously (e.g., concurrently) as either separate therapies/formulations or as a joint therapy/formulation. Alternatively, the therapies or agents can be administered sequentially, as separate compositions, within an appropriate time frame as determined by the skilled clinician (e.g., a time sufficient to allow an overlap of the pharmaceutical effects of the therapies). The retinoic acid or derivative thereof and one or more additional therapies or therapeutic agents can be administered in a single dose or in multiple doses, in an order and on a schedule suitable to achieve a desired therapeutic effect. Suitable dosages and regimens of administration can be determined by a clinician and are dependent on the agent(s) chosen, pharmaceutical formulation and route of administration, various patient factors and other considerations.

A retinoic acid or derivative thereof can be administered to a mammalian subject in a pharmaceutical or physiological composition, for example, as part of a pharmaceutical composition comprising a retinoic acid or derivative thereof and a pharmaceutically acceptable carrier. Formulations or compositions (e.g., solution, emulsion or capsule) comprising a retinoic acid or derivative thereof or compositions comprising a retinoic acid or derivative thereof and one or more other therapeutic agents (e.g.,) will vary according to the route of administration selected.

Suitable pharmaceutical carriers can contain inert ingredients which do not interact with the retinoic acid or derivative thereof. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying, solubilizing, pH buffering, wetting agents). Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

A retinoic acid or derivative thereof may be administered to a subject by a variety of routes, preferably in the form of a pharmaceutical composition adapted to a desired route. The preferred route will depend, in part, on the condition being treated. The compounds and compositions may, for example, be administered intravascularly, intramuscularly, subcutaneously, intraperitoneally, orally or topically. It will be obvious to those skilled in the art that the following dosage forms may comprise as the active ingredient, either compounds or a corresponding pharmaceutically acceptable salt of a compound. Preferred routes of administration for the retinoic acid or derivatives thereof include oral and intravenous administration.

A retinoic acid or derivative thereof may be administered parenterally, via injection. Parenteral administration can include, for example, intraarticular, intramuscular, intravenous, intraventricular, intraarterial, intrathecal, subcutaneous, or intraperitoneal administration. Formulations for parenteral administration may be in the form of aqueous or non-aqueous isotonic sterile injection solutions or suspensions. These solutions or suspensions may be prepared from sterile powders or granules having one or more of the carriers mentioned for use in the formulations for oral administration. The compounds may be dissolved in polyethylene glycol, propylene glycol, ethanol, corn oil, benzyl alcohol, sodium chloride, and/or various buffers (e.g., sodium bicarbonate, sodium hydroxide).

For oral administration, the pharmaceutical compositions comprising a retinoic acid or derivative thereof may be in the form of, for example, a tablet, capsule, suspension or liquid. The composition is preferably made in the form of a dosage unit containing a therapeutically effective amount of the active ingredient. Examples of such dosage units are tablets and capsules. For therapeutic purposes, the tablets and capsules can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

The invention further provides a method of preserving a kidney (e.g., a human kidney) for transplant (e.g., cold organ preservation), comprising contacting the kidney with a solution that comprises a retinoic acid or a retinoic acid derivative. Preferably, the retinoic acid or retinoic acid derivative is 9-cis-retinoic acid or 13-cis-retinoic acid. The solution comprising the retinoic acid or derivative thereof may include one or more additional components, such as, for example, dexamethasone, insulin, penicillin G, glutathione, allopurinol, or a combination thereof.

A description of example embodiments of the invention follows.

### Exemplification

### Mice

C57B6/J and Nur77 knockout mice were purchased from Jackson laboratories. Animals were housed in the AALAC-approved animal facility in a pathogen free environment. All the animal studies were approved by the institutional review board at the Beth Israel Deaconess Medical Center.

### Ischemia Reperfusion Injury

Male mice of age 8-10 weeks were used in this study. Animals were anesthetized using xylazine and ketamine and kept on 37° C. warming pads. The kidneys were exposed by flank incision and both kidney pedicles were clamped for 45 minutes using non-traumatic microaneurysm clamps (Roboz, Rockville, Md.). Reperfusion was confirmed visually and the incision was sutured. Body temperature of the animals was maintained at 37° C. throughout the procedure.

### 9-Cis Retinoic Acid Administration

9-cis retinoic acid in DMSO was administered to animals at a dose of 10 mg/kg body weight, 4 h prior to the IRI procedure. The final concentration of DMSO was 25% (v/v). Control animals received 25% (v/v) DMSO.

### Renal Histopathology and Immunohistochemistry

Kidneys from animals subjected to IRI were harvested. Transverse sections of the kidneys were either snap frozen in OCT or fixed in 10% formalin and processed for paraffin embedded sections. Renal morphology was assessed by PAS staining (Periodic acid-Schiff reagent). Neutrophil infiltration was assessed by staining formalin fixed paraffin embedded (FFPE) sections with anti-Ly6G (BD). Infiltrating neutrophils were counted in 3 independent HPFs (400×) per section per animal and represented as mean±SEM. Nur77 staining was carried out on FFPE sections after antigen retrievel using anti-mouse Nur77 antibody (Ebioscience). Endogenous mouse Ig was blocked using M.O.M kit from vector labs.

### TUNEL Staining

Kidney cell apoptosis was assessed by TUNEL staining using ApopTag® Peroxidase In Situ Apoptosis Detection Kit (Millipore). The apoptotic index was calculated as percentage of dead cells in 3 independent 400×HPF per section per animal by, counting both stained nuclei and total nuclei.

### Analysis of Kidney Function

Serum plasma was collected 24 h after ischemia reperfusion and stored at −80 deg C. until further processing. Serum creatinine was quantified using QuantiChrom™ Creatinine Assay Kit (Bioassays systems). Serum blood urea nitrogen levels were measured using Infinity Urea liquid stable reagent (Thermo fisher).

### Real Time PCR Quantification

Kidney tissues were snap frozen and stored at −80 deg C. until processing. The tissues were homogenized and total RNA was prepared using RNeasy kit (Qiagen). DNA contamination was eliminated by DNase digestion. The reverse transcribed cDNA was subjected to qPCR using a Sybr green based detection system (SA Bioscience). Relative levels of mRNA was normalized to GAPDH levels and quantified based on 2e-deltaCT method.

### Cell Line and Chemical Induced IRI

Human Kidney Proximal tubular epithelial cell line, HK2 was purchased from ATCC and maintained in Keratinocyte-Serum Free Medium (K-SFM) at 37 deg C. with 5% CO2. To perform chemical induced IRI, cells were washed with Hank's Buffered Salt Solution (HBSS) and incubated with HBSS containing Antimycin (10 uM), 2-Deoxy Glucose (10 mM) and A23187 (2 uM) for 1 hour at 37 deg C., 5% CO2. Subsequently the cells were washed with HBSS and cultured in complete culture medium for defined period of time.

### RNA In Situ Hybridization

Nur77 RNA expression was localized by RNA in situ hybridization following the protocol as described elsewhere³³. Nur77 riboprobe corresponds to 300 by nucleotides complementary to 581-880 by of the Nur77 coding sequence.

### Mouse Primary Renal Proximal Tubule Culture

Kidneys from 4-5 weeks old mice were harvested after perfusion with sterile PBS. Cortex was separated and minced into ˜1 mm3 pieces and digested using collagenase and soybean trypsin inhibitor for 30 min and 37 degC. The digested mix was spun down and filtered through 200 uM filter followed by a 70 uM filter. The tubules that are retained on the top of the 70 uM filter were collected and cultured in Renal life cell growth medium (Lifeline cell technology) on collagen coated plates. After 7 days of culturing, the cells were used for experiments. The purity of the culture was ascertained by staining for cytokeratin (Anti-cytokeratin antibody: Sigma). To induce ischemia reperfusion injury, confluent monolayer of RPTECs was overlayed with mineral oil for 1 h, followed by reperfusion with complete medium.

### Statistical Analysis

All comparisons were performed using a two-tailed unpaired Student's t test.

### Results

### NR4a Family Members are Rapidly Induced in Murine Ischemia-Reperfusion Injury

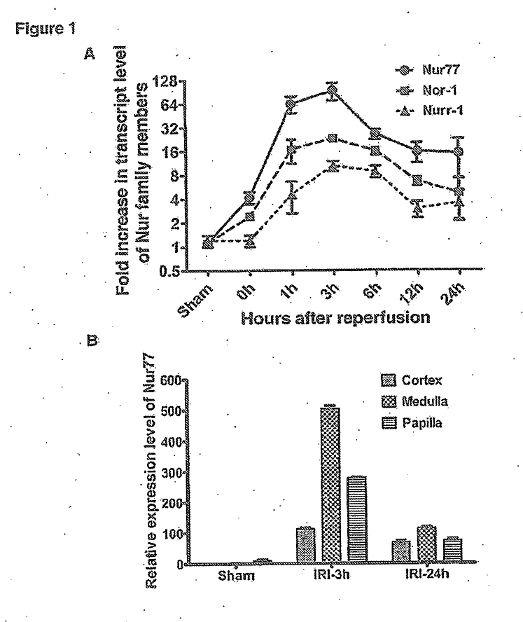

The observation that NR4a members are induced at the mRNA level in whole kidney lysates either by parathyroid hormone injection²⁰ or in a glomerular disease model²¹ prompted us to examine a possible role for NR4a family members in acute kidney injury. Using the murine IRI model of acute kidney injury, the expression of Nur77, Nurr1 and Nor-1 was examined during the early phases of AKI. Nur77 was very rapidly and strongly induced after renal IRI, with a lower induction of both Nor-1 and Nurr1 (FIG. 1A). mRNA levels were maximal three hours after reperfusion with falling levels for all three NR4a family members thereafter. Renal damage is most severe in kidney outer medulla in the IRI model, and to determine regional expression of Nur77 after injury, qPCR was performed on kidney cortex, medulla and papilla after IRI. While Nur77 was induced in all three regions, expression was highest in medulla at both 3 hours and 24 hours after IRI, consistent with a role for Nur77 in acute response to injury in outer medulla (FIG. 1B).

### Nur77 Expression is Induced in Proximal Tubule Epithelia In Vitro and In Vivo

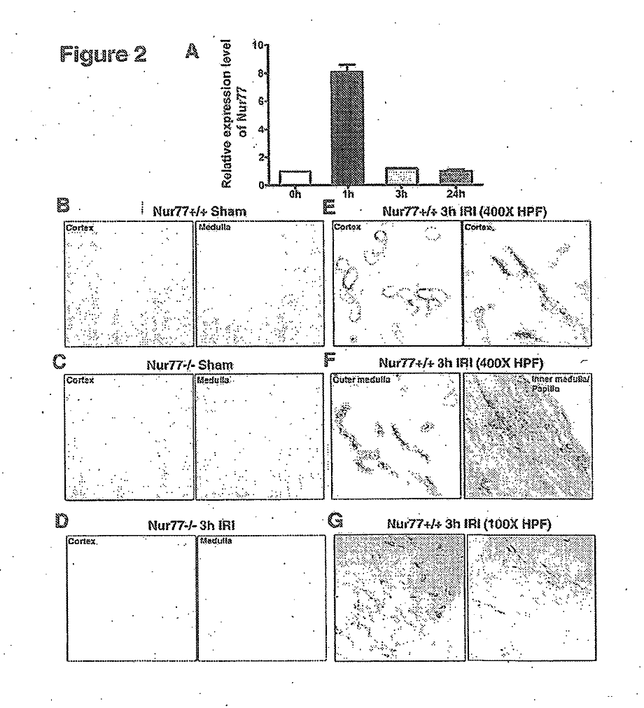

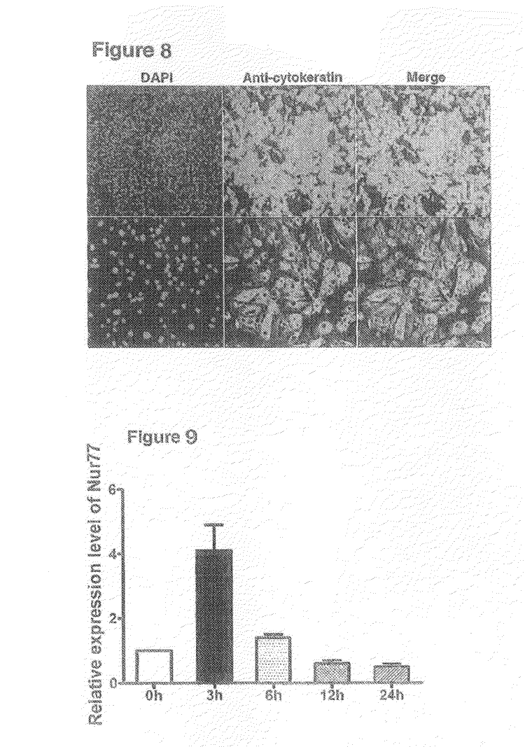

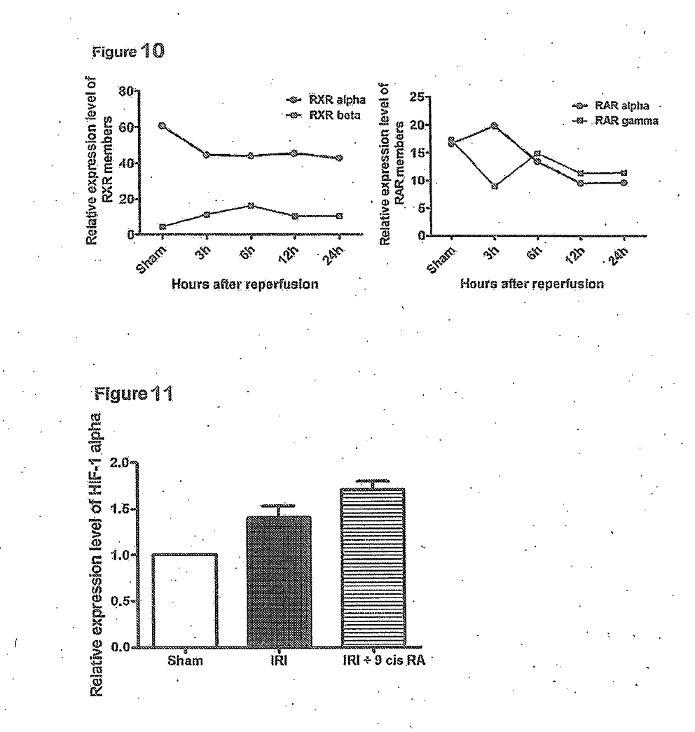

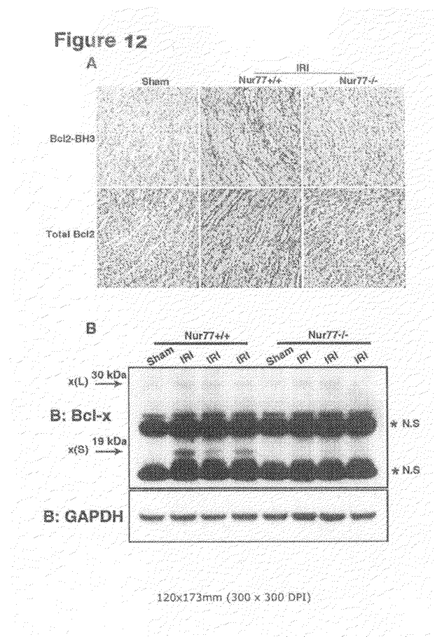

The renal cell type expressing Nur77 after acute injury was explored. To assess whether Nur77 is capable of being induced in proximal tubule, the major tubule segment present in outer medulla, primary proximal tubule kidney cultures (FIG. 8) were subject to hypoxia in vitro by mineral oil overlay. Nur77 was rapidly induced one hour after hypoxia, but levels returned to baseline by three hours (FIG. 2A). In the immortalized human proximal tubule cell line HK2, similar results were found. A chemical ischemia protocol rapidly induced Nur77 mRNA within three hours of treatment, with levels falling back to baseline thereafter (FIG. 9). Together, these observations suggest that Nur77 is capable of being induced by hypoxia in proximal tubule epithelia. The more rapid resolution of Nur77 expression in vitro compared to in vivo may reflect less severe injury, more rapid normalization of oxygen tension or the lack of an inflammatory component in vitro.

To define the identity of cells that express Nur77 mRNA after acute kidney injury in vivo, kidneys from wildtype or Nur77 null mice were subject to in situ hybridization for Nur77 message after renal injury. As expected, Nur77 was undetectable in sham-operated kidneys. Three hours after renal IRI, Nur77 signal was present in cortical tubules that showed evidence of damage, in medullary tubules and in papilla (FIG. 2B-G). The dilated appearance of some of these tubules are consistent with the appearance of injured proximal tubules at this stage of IRI. As a negative control, kidneys from Nur77 knockout mice subjected to the same injury had undetectable Nur77 by in situ hybridization after renal injury (FIG. 2C, D). Thus, acute injury induces Nur77 expression in proximal tubule epithelia both in vitro and in vivo.

### Nur77 Exacerbates Renal Injury and Promotes Epithelial Apoptosis

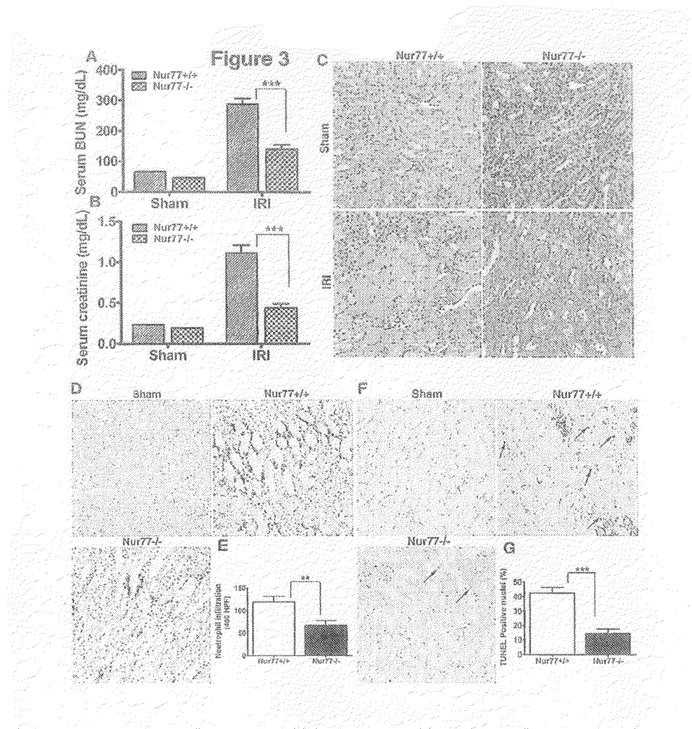

To examine the functional role of Nur77 in renal IRI, the effect of renal IRI between wild type and Nur77 knockout mice which have no detectable phenotype at baseline was compared 22. 24 hours after injury, wild type mice had substantially worse renal function than Nur77− littermate controls as reflected by higher serum BUN and creatinine values (FIG. 3A, B). In addition, renal histology was characterized by more intense tubular damage in wild type mice compared to Nur77-mice, with numerous tubules full of necrotic debris and simplified, dedifferentiated epithelium in wild type kidneys (FIG. 3C).

Since epithelial apoptosis is an important consequence of ischemic injury and Nur77 can be proapoptotic in many cellular contexts, tubular apoptosis was next assessed by TUNEL staining. There was a significant reduction of TUNEL-positive tubular nuclei in Nur77− kidneys compared to controls by 50% (FIG. 3D, E), suggesting that Nur77 normally functions to promote renal apoptosis in proximal tubule after damage. Nur77− mice also exhibited a 50% reduction in interstitial neutrophil accumulation (FIG. 3F, G). Together, these results indicate that induction of Nur77 in renal epithelia after acute injury causes apoptosis leading to worse renal function, histologic damage and neutrophil recruitment.

Reduced Proinflammatory Mediators in Nur77 Deficient Mice after Acute Kidney Injury

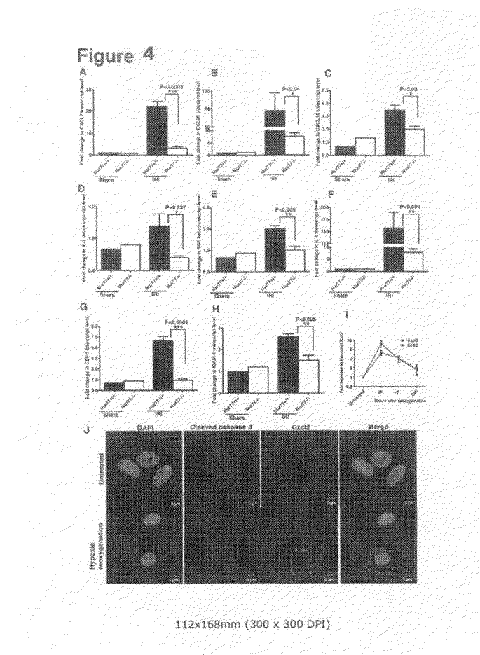

Epithelial apoptosis after renal IRI is a pro-inflammatory stimulus through elaboration of soluble mediators that activate and recruit leukocytes²³. Since previous results indicated that Nur77 promotes epithelial apoptosis and neutrophil accumulation, it was hypothesized that Nur77-deficient mice that are characterized by reduced epithelial apoptosis would also elaborate fewer proinflammatory cytokines. Levels of these cytokines were assessed by qPCR. mRNA encoding CCL20, CXCL10, CSF-1, IL-6 and TGF β, all cytokines capable of being secreted by damaged epithelia were substantially inhibited in Nur77 deficient kidney lysates (FIG. 4). CXCL2 was also suppressed nearly to baseline levels, as was the epithelial toll-like receptor TLR4 and the adhesion receptor ICAM-1. These findings are consistent with the notion that epithelial apoptosis after AKI is pro-inflammatory, and that the absence of Nur77 reduces both apoptosis and resultant inflammation.

These results, however, do not distinguish between Nur77-dependent promotion of apoptosis and Nur77-mediated stimulation of inflammation. To distinguish between these possibilities we examined the expression pattern of Cxcl2, a chemokine that is significantly diminished in the Nur77 null mice (FIGS. 4A and D), in an in vitro model of ischemia reperfusion injury. Cxcl2 gene expression was noted as early as 1 h post reperfusion/reoxygenation of primary cultures of renal proximal tubular epithelial cells, which is devoid of other cell types (FIG. 41). There was no evidence of apoptosis (nuclear condensation or cleaved caspase-3) at this time point, indicating that Cxcl2 is not simply induced in epithelial cells fated to die (FIG. 4J).

### Nur77 Mediates Renal Injury Via Co-Opting Bcl2 Family Proteins

Nur77 dependent apoptotic induction in cells can convert pro-survival Bcl2 to a proapoptotic molecule by exposing its BH3 domain via conformational change. Immunostaining of kidney tissues with a specific anti-BH3 domain antibody demonstrated pronounced exposure of the BH3 domain of Bcl2 in wild type, but not Nur77 KO or sham operated kidneys (FIG. 12A). Further, we identified the involvement of another Bcl2 family protein, Bcl-xS, the alternative spliced variant of Bcl21, a well characterized pro-apoptotic molecule, in Nur77 mediated kidney injury (FIG. 12B). Upon IRI, Bcl-xS protein levels were elevated in wild type kidneys as compared to Nur77 KO kidneys. Thus Nur77 apparently mediates renal epithelial apoptosis via the Bcl2 pathway.

### Retinoic Acid Receptor Ligand Inhibits the Epithelial Stress Response in a Nur77-Dependent Fashion

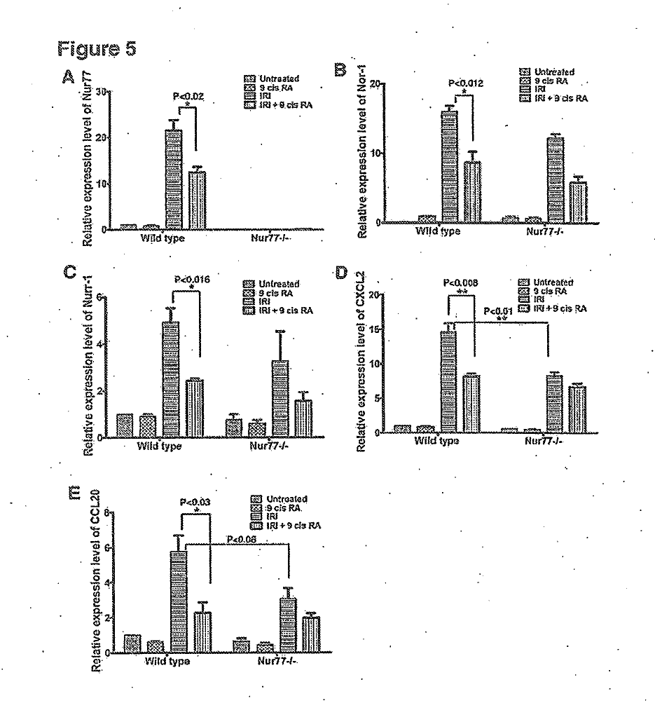

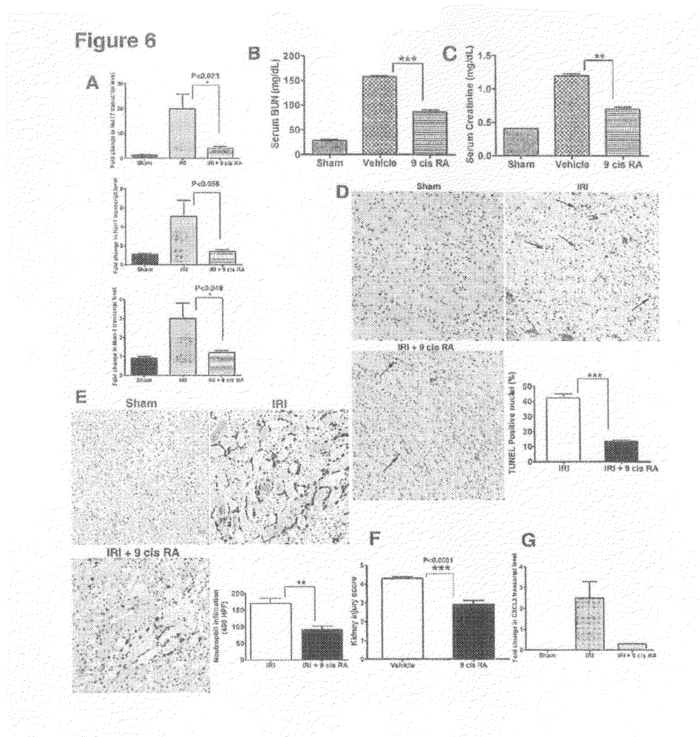

Retinoic acid can antagonize Nur77 function by inhibiting its transcriptional induction, DNA binding and nuclear export^{17,18}. RXRs heterodimerize with Nur77 in response to cell death stimuli, promoting nuclear export and causing Bcl-2 dependent cytochrome c release and apoptosis. Ligand bound RXR inhibits Nur77 function either by retaining it in the nucleus or by inhibiting its transcription by an AP-1 dependent mechanism¹⁸. Since Nur77 deficient mice were protected from renal IRI, whether pharmacologic antagonism of Nur77 by retinoic acid could be used to protect kidneys from acute injury was assessed. To determine whether retinoids could regulate Nur77-dependent signaling in renal epithelia, primary cultures of renal proximal tubule epithelial cells (RPTECs) were generated from either wild type or Nur77 knockout mice. RPTECs were treated with either vehicle or 9-cis-retinoic acid, and subjected to mineral oil overlay induced hypoxia, followed by reperfusion. Nur77 levels were reduced by about half in 9-cis-retinoic acid-treated cultures (FIG. 5A). Induction of Nr4a family members was also inhibited by 9-cis-retinoic acid treatment, both in wild type and Nur77 knockout cultures (FIG. 5B, C). CXCL2 was induced by hypoxia, and 9-cis-retinoic acid significantly attenuated induction of CXCL2 in RPTECs. In cells from Nur77 knockout kidneys, however, 9-cis-retinoic acid had no further inhibitory effect, indicating that inhibition was Nur77 dependent (FIG. 5D). Similarly, CCL20 levels were very substantially reduced by 9-cis-retinoic acid treatment, whereas CCL20 induction was reduced in Nur77 knockout RPTECs, with no further effect of 9-cis retinoic acid (FIG. 5E). These results provide direct evidence that in RPTECs, retinoids regulate the stress response by a Nur77-dependent mechanism.

Nur77 Antagonism with Retinoic Acid Receptor Ligand Ameliorates Acute Kidney Injury

To investigate whether 9-cis-retinoic acid would protect from renal IRI, mice were treated with vehicle 9-cis retinoic acid, four hours prior to renal IRI. 24 hours after injury, mice that received 9-cis retinoic acid had dramatically lower induction of all three NR4a members Nur77, Nor-1 and Nurr-1 (FIG. 5A), consistent with the in vitro results. At the same time point, treated mice had reduced serum creatinine and BUN levels, reflecting better renal function (FIG. 5B, C). Since Nur77 deficient mice had reduced apoptosis after renal IRI, whether 9-cis retinoic acid reduced apoptosis in wild type mice was also investigated. Consistent with previous results with Nur77 deficient mice, there was significantly less Tuner apoptotic nuclei in 9-cis retinoic acid-treated mice, and much less neutrophil infiltration and improved renal injury scores (FIG. 5D-F). Reduced CXCL2 transcript was detected in 9-cis retinoic acid treated mice, in support of the in vitro findings (FIG. 5G). These results indicate that 9-cis retinoic acid is protective in renal IRI and suggest that this protection is mediated by repression of Nur77-induced apoptosis and inflammation.

### Retinoic Acid Inhibits Nur77 Induction but not Subcellular Localization in Acute Kidney Injury

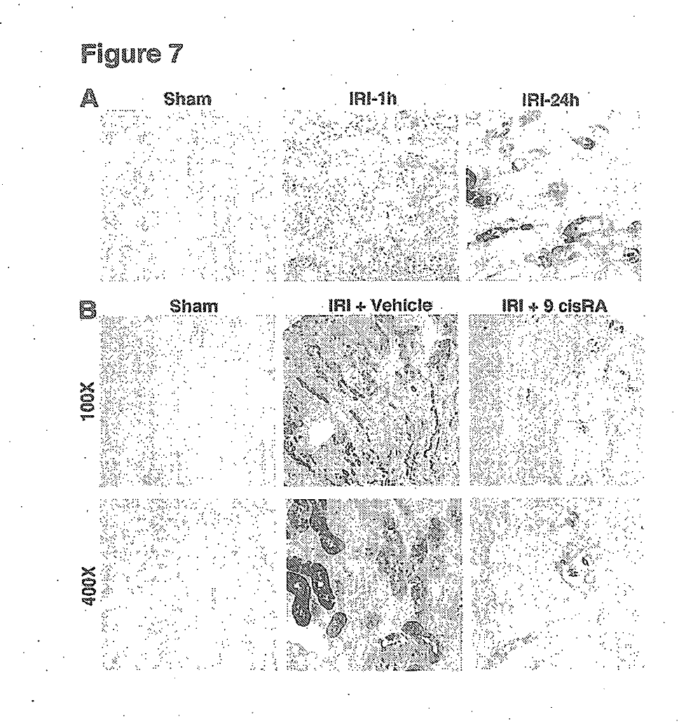

Since retinoids are capable of inhibiting Nur77 by several mechanisms including both inhibition of transcriptional induction and preventing nuclear to cytoplasmic translocation, the Nur77 protein expression and subcellular localization in injured kidney was investigated. While Nur77 was undetectable in uninjured kidneys, one hour after renal IRI Nur7 could be detected in a nuclear pattern in epithelial cells. By 24 hours after injury, Nur77 expression was cytoplasmic, consistent with its known proapoptotic effects in cytoplasm (FIG. 7A). In mice that received 9-cis-retinoic acid, the overall level of Nur77 protein expression was dramatically reduced, in agreement with mRNA levels. At 24 hours after injury, even in mice that received 9-cis retinoic acid, Nur77 was cytoplasmic—suggesting that in renal epithelia, retinoids act predominantly to inhibit Nur77 transcriptional induction, rather than to promote its nuclear retention (FIG. 7B).

### Discussion

Acute kidney injury remains a major health problem with few therapeutic options. The present study provides the first demonstration that Nur77 is rapidly and strongly induced in injured proximal tubule epithelia where it regulates apoptosis. Epithelial apoptosis after injury triggers a pro-inflammatory cascade resulting in tissue damage, and inhibition of apoptosis is a protective therapeutic strategy in preclinical and clinical settings²³⁻²⁵. These findings implicate Nur77 as a key regulator of both epithelial apoptosis but also of inflammation caused by injury-induced cell death. Furthermore, these results identify 9-cis retinoic acid as a protective therapy in acute kidney injury through inhibition of Nur77 induction, providing a novel therapeutic target for treatment of acute kidney injury.

Nur77 was induced in proximal tubule epithelial cells based on two lines of evidence. Primary cultures of proximal tubule epithelia were capable of inducing Nur77 expression after hypoxia, and Nur77 mRNA was found in injured proximal tubules by in situ hybridization. These results are consistent with an earlier observation that systemic parathyroid hormone injection causes increased kidney expression of Nur77²⁰, because proximal tubules express the parathyroid hormone receptor²⁶. The induction of Nur77 in proximal tubules, the nephron segment most susceptible to hypoxic insult, suggests a role for Nur77 in mediating the stress response in injured epithelia.

The ability to pharmacologically antagonize Nur77 with retinoids represents an attractive therapeutic strategy. Retinoic acid has been shown to protect from toxicant-induced renal injury¹⁹, but the mechanism is unknown. These results suggest that in kidney, retinoids repress stress-induced Nur77 transcriptional induction. Retinoic acid is known to inhibit transcriptional induction of Nur77 by an AP-1-dependent mechanism in lymphocytes¹⁸, consistent with the observation of reduced expression of Nur77 mRNA and protein after retinoid treatment both in vitro and in vivo. In addition, Nur77 is capable of heterodimerization with ligand-bound retinoic acid receptor¹⁸, which prevents its nuclear export. Nuclear retention of Nur77 prevents its translocation to mitochondrial and cytochrome c release, preventing apoptosis^{13,17}. RXR is a critical regulator of Nur77 translocation and apoptosis in response to a variety of extracellular ligands and stimuli (reviewed in ²⁷), and the RXR nuclear export signal is conformationally regulated by retinoic acid ligand¹⁷. However, no change was seen in the pattern of nuclear to cytoplasmic Nur77 localization in kidney after 9-cis retinoic acid treatment, suggesting that retinoids act solely to repress Nur77 induction rather than to regulate its nuclear export. Combined with evidence that anti-apoptotic strategies can be therapeutic in other preclinical models of ischemia-reperfusion injury²⁸⁻³⁰, these results provide a basis for further exploration of the therapeutic potential of Nur77-targeted therapies in acute kidney injury.

The mechanism by which Nur77 expression in tubule epithelia triggers apoptosis requires further study, but may involve Bcl-2. While it typically functions as a survival factor, Bcl-2 is a mitochondrial receptor for Nur77. This interaction triggers a conformational change in Bcl-2, exposing its pro-apoptotic BH3 domain, causing cytochrome c release and apoptosis¹⁶. BH3-only proteins exert proapoptotic effects through either Bax or Bak³¹, and indeed Nur77-dependent apoptosis is dependent on Bak in lung cancer cells¹⁶. Bax is proapoptotic in renal epithelial cells, where it is regulated by cell survival kinases Akt and glycogen synthase kinase 3β (GSK3β)³². The finding that Nur77 expression promotes epithelial apoptosis, and that Nur77 translocates from nucleus to cytoplasm during renal injury is consistent with a model involving Bcl-2-dependent proapoptotic signaling. Whether Nur77-dependent apoptosis in renal epithelial cells depends on multidomain proapoptotic Bcl-2-family proteins like Bax or Bak requires further investigation.

In summary, the data shown herein indicate a novel role for Nur77 in promoting epithelial apoptosis and inflammation after acute kidney injury. The ability to antagonize Nur77 function with retinoic acid ligands and thereby ameliorate ischemic renal injury represents a novel therapeutic strategy for this important clinical syndrome.

### REFERENCES

1. Chertow, G. M., Burdick, E., Honour, M., Bonventre, J. V. & Bates, D. W. Acute kidney injury, mortality, length of stay, and costs in hospitalized patients. J Am Soc Nephrol 16, 3365-3370 (2005).
2. Lassnigg, A., et al. Minimal changes of serum creatinine predict prognosis in patients after cardiothoracic surgery: a prospective cohort study. J Am Soc Nephrol 15, 1597-1605 (2004).
3. Ympa, Y. P., Sakr, Y., Reinhart, K. & Vincent, J. L. Has mortality from acute renal failure decreased? A systematic review of the literature. Am J Med 118, 827-832 (2005).
4. Humphreys, B. D., et al. Intrinsic epithelial cells repair the kidney after injury. Cell Stem Cell 2, 284-291 (2008).
5. Schumer, M., et al. Morphologic, biochemical, and molecular evidence of apoptosis during the reperfusion phase after brief periods of renal ischemia. Am J Pathol 140, 831-838 (1992).
6. Padanilam, B. J. Cell death induced by acute renal injury: a perspective on the contributions of apoptosis and necrosis. Am J Physiol Renal Physiol 284, F608-627 (2003).
7. Choi, J. W., Park, S. C., Kang, G. H., Liu, J. O. & Youn, H. D. Nur77 activated by hypoxia-inducible factor-1alpha overproduces proopiomelanocortin in von Hippel-Lindau-mutated renal cell carcinoma. Cancer Res 64, 35-39 (2004).
8. Rosenberger, C., et al. Expression of hypoxia-inducible factor-1 alpha and -2alpha in hypoxic and ischemic rat kidneys. J. Am. Soc Nephrol 13, 1721-1732 (2002).
9. Yoo, Y. G., et al. 6-Mercaptopurine, an activator of Nur77, enhances transcriptional activity of HIF-1alpha resulting in new vessel formation. Oncogene 26, 3823-3834 (2007).
10. Hazel, T. G., Nathans, D. & Lau, L. F. A gene inducible by serum growth factors encodes a member of the steroid and thyroid hormone receptor superfamily. Proc Natl Acad Sci USA 85, 8444-8448 (1988).
11. Dequiedt, F., et al. HDAC7, a thymus-specific class II histone deacetylase, regulates Nur77 transcription and TCR-mediated apoptosis. Immunity 18, 687-698 (2003).
12. Li, Y., et al. Molecular determinants of AHPN (CD437)-induced growth arrest and apoptosis in human lung cancer cell lines. Mol Cell Biol 18, 4719-4731 (1998).
13. Li, H., et al. Cytochrome c release and apoptosis induced by mitochondrial targeting of nuclear orphan receptor TR3. Science 289, 1159-1164 (2000).
14. Wilson, A. J., Arango, D., Mariadason, J. M., Heerdt, B. G. & Augenlicht, L. H. TR3/Nur77 in colon cancer cell apoptosis. Cancer Res 63, 5401-5407 (2003).
15. Kolluri, S. K., et al. A short Nur77-derived peptide converts Bcl-2 from a protector to a killer. Cancer Cell 14, 285-298 (2008).
16. Lin, B., et al. Conversion of Bcl-2 from protector to killer by interaction with nuclear orphan receptor Nur77/TR3. Cell 116, 527-540 (2004).
17. Cao, X., et al. Retinoid X receptor regulates Nur77/TR3-dependent apoptosis [corrected] by modulating its nuclear export and mitochondrial targeting. Mol Cell Biol 24, 9705-9725 (2004).
18. Kang, H. J., et al. Retinoic acid and its receptors repress the expression and transactivation functions of Nur77: a possible mechanism for the inhibition of apoptosis by retinoic acid. Exp Cell Res 256, 545-554 (2000).
19. Perez, A., et al. Beneficial effect of retinoic acid on the outcome of experimental acute renal failure. Nephrol Dial Transplant 19, 2464-2471 (2004).
20. Pirih, F. Q., Aghaloo, T. L., Bczouglaia, O., Nervina, J. M. & Tetradis, S. Parathyroid hormone induces the NR4A family of nuclear orphan receptors in vivo. Biochem Biophys Res Commun 332, 494-503 (2005).
21. Hayashi, K., Ohkura, N., Miki, K., Osada. S. & Tomino, Y. Early induction of the NGFI-B/Nur77 family genes in nephritis induced by anti-glomerular basement membrane antibody. Mol Cell Endocrinol 123, 205-209 (1996).
22. Lee, S. L., et al. Unimpaired thymic and peripheral T cell death in mice lacking the nuclear receptor NGFI-B (Nur77). Science 269, 532-535 (1995).
23. Daemen, M. A., et al. Inhibition of apoptosis induced by ischemia-reperfusion prevents inflammation. J Clin Invest 104, 541-549 (1999).
24. Kelly, K. J., et al. Minocycline inhibits apoptosis and inflammation in a rat model of ischemic renal injury. Am J Physiol Renal Physiol 287, F760-766 (2004).
25. Valentino, K. L., Gutierrez, M., Sanchez, R., Winship, M. J. & Shapiro, D. A. First clinical trial of a novel caspase inhibitor: anti-apoptotic caspase inhibitor, IDN-6556, improves liver enzymes. Int J Clin Pharmacol Ther 41, 441-449 (2003).
26. Ba, J., Brown, D. & Friedman, P. A. Calcium-sensing receptor regulation of PTH-inhibitable proximal tubule phosphate transport. Am J Physiol Renal Physiol 285, F1233-1243 (2003).
27. Zhang, X. K. Targeting Nur77 translocation. Expert Opin Ther Targets 11, 69-79 (2007).
28. Hochhauser, E., et al. Bax ablation protects against myocardial ischemia-reperfusion injury in transgenic mice. Am J Physiol Heart Circ Physiol 284, H2351-2359 (2003).
29. Hetz, C., et al. Bax channel inhibitors prevent mitochondrion-mediated apoptosis and protect neurons in a model of global brain ischemia. J Biol Chem 280, 42960-42970 (2005).
30. Hamar, P., et al. Small interfering RNA targeting Fas protects mice against renal ischemia-reperfusion injury. Proc Natl Acad Sci USA 101, 14883-14888 (2004).
31. Wei, M. C., et al. Proapoptotic BAX and BAK: a requisite gateway to mitochondrial dysfunction and death. Science 292, 727-730 (2001).
32. Wang, Z., et al. Beta-catenin promotes survival of renal epithelial cells by inhibiting Bax. J Am Soc Nephrol 20, 1919-1928 (2009).
33. Brunskill, E. W., et al. Atlas of gene expression in the developing kidney at microanatomic resolution. Dev Cell 15, 781-791 (2008).

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of inhibiting apoptosis in kidney epithelial cells after acute injury by modulating/inhibiting Nur77 induction/expression or function/activity in the cells.

2. The method of claim 1 wherein the kidney cells are proximal tubule epithelial cells.

3. The method of claim 1 wherein the acute injury is a hypoxia-induced injury.

4. The method of claim 1 wherein the hypoxia is an ischemic injury.

5. The method of claim 1 wherein Nur77 induction/expression is inhibited by contacting the cells with retinoic acid, or a derivative thereof.

6. The method of claim 5 wherein the retinoic acid, or derivative thereof, is a cis-retinoic acid.

7. The method of claim 6 wherein the cis-retinoic acid is 9-cis-retinoic acid or 13-cis-retinoic acid.

8. A method of preventing or treating an acute kidney injury in a subject in need thereof, comprising administering to the subject an effective amount of a retinoic acid or retinoic acid derivative.

9. The method of claim 8, wherein the retinoic acid or retinoic acid derivative is 9-cis-retinoic acid or 13-cis-retinoic acid.

10. The method of claim 8, wherein the acute kidney injury is a hypoxia-induced injury.

11. The method of claim 8, wherein the acute kidney injury is an ischemic injury.

12. The method of claim 8, wherein the acute kidney injury is a toxicant-induced injury or a septic injury.

13. The method of claim 8, wherein the retinoic acid or retinoic acid derivative is administered in combination with one or more therapies selected from the group consisting of vasodilation, volume replacement, blood purification and renal replacement therapy.

14. The method of claim 8, wherein the subject is a human.

15. The method of claim 8, wherein the retinoic acid or retinoic acid derivative is administered orally.

16. The method of claim 8, wherein the retinoic acid or retinoic acid derivative is administered intravenously.

17. A method of preserving a kidney for transplant, comprising contacting the kidney with a solution that comprises a retinoic acid or a retinoic acid derivative.

18. The method of claim 17, wherein the solution additionally comprises one, or more of the following components: dexamethasone, insulin, penicillin G, glutathione, allopurinol or a combination thereof.

19. The method of claim 17, wherein the retinoic acid or retinoic acid derivative is 9-cis-retinoic acid or 13-cis-retinoic acid.

20. The method of claim 17, wherein the kidney is subjected to cold organ preservation.

21. The method of claim 17, wherein the kidney is a human kidney.
